(19) United States
(12) Patent Application Publication
(10) Pub. No.: US 2025/0325246 A1
(43) Pub. Date: Oct. 23, 2025

(54) ULTRASONIC ROD PROBE

(71) Applicant: PROCEPT BioRobotics Corporation, San Jose, CA (US)

(72) Inventors: Kurt RUFFIEUX, Horgen (CH); Arkadiusz MIERNIK, Freiburg (DE)

(73) Assignee: PROCEPT BioRobotics Corporation, San Jose, CA (US)

(21) Appl. No.: 18/861,781

(22) PCT Filed: Apr. 27, 2023
(86) PCT No.: PCT/EP2023/061116

§ 371 Date: Apr. 23, 2025

(30) Foreign Application Priority Data
Apr. 27, 2022 (DE) 10 2022 110 203.3

(51) Int. Cl. A61B 8/12; A61B 8/00

(52) U.S. Cl. CPC A61B 8/12; A61B 8/4254; A61B 8/429; A61B 8/445

(57) ABSTRACT

Described is an arrangement with an ultrasonic rod probe for carrying out rectal or vaginal examinations, having a distally rounded probe head, at least in which an ultrasonic transducer is integrated. The invention is distinguished by the fact that attached to the probe head is at least one pressure-sensitive element which, in response to mechanical contact with an object, undergoes deformation when a pressure acting between the pressure-sensitive element and the object is exceeded.

### TECHNICAL FIELD

The invention relates to an ultrasonic rod probe for performing rectal or vaginal examinations having a distally rounded probe head in which at least one ultrasound transducer is integrated.

A common clinical picture in urology concerns the enlargement of the prostate in men (BPH, benign prostatic hyperplasia). The enlargement of the prostate gland causes a narrowing of the urethra, which can lead to difficult urination or complete urinary retention (ischuria). In the early stages of benign prostatic hyperplasia. medication-based treatment options are available; in more advanced BPH, which is associated with pronounced symptoms or complications, surgical interventions usually provide relief.

Various surgical methods can be used as part of prostate treatment at an advanced stage. One of these methods uses the abrasive effect of a water jet, in which the benign hyperplasia (adenoma) on the prostate is removed using a focused high-pressure water jet. To determine the shape and size of the adenoma to be removed, the surgeon inserts an ultrasound probe rectally and guides it to the site of the prostate.

Image processing software calculates the region to be treated on the basis of the images obtained with the ultrasound probe and controls the therapeutically effective water jet. The surgeon must readjust the ultrasound probe several times during the surgical preparation, i.e. rotate the ultrasound probe, move it distally and/or proximally and/or laterally/medially.

During the insertion of the ultrasound probe, as well as during the subsequent advancement or retraction of the ultrasound probe, there is a risk of injuring the bowel, especially if the ultrasound probe is in contact with a crease in the bowel. A rupture of the bowel wall caused thereby can have various effects: a) Injury to the bowel wall with a breach into the peritoneal space can lead to serious infections and even sepsis. including death, b) An injury in the region of the pararectal/paravesical space can lead to local symptoms, e.g. a painful abscess that can take several months to heal.

Such injuries must be avoided during examination and handling with ultrasound probes.

### PRIOR ART

Publication DE 69913936 T2 describes an ultrasonic probe with a detachable needle guide for taking tissue samples. which provides two raster ultrasonic transducers on the distal. dome-shaped probe head, through which a hollow channel also runs, the longitudinal axis of which is oriented at an angle to the longitudinal axis of the ultrasonic probe and which is used to insert a biopsy needle.

DE 39 29 611 A1 discloses an intracavitary ultrasonic probe, in particular for use in rectal examinations. at the distal end of which is arranged an ultrasonic transducer which is movably mounted about two spatial axes and which can be spatially pivoted via manually operable drive axes. The ultrasonic transducer is otherwise surrounded by an ultrasound-conducting housing which has a rounded surface.

EP 0 589 082 B1 discloses a prostate probe for generating sectional images of the examination region. A swivel-mounted, convex transducer array in the form of a semicircle is attached to the distal probe region.

EP 2 403 603 B1 discloses a transurethral ultrasound probe for treating the prostate with an ultrasound transducer arrangement mounted to rotate in the longitudinal direction of the probe within a probe housing, with a cooling and acoustic coupling fluid flowing through the interior of the probe for acoustic coupling to the probe housing.

EP 2 872 045 B1 discloses an ultrasonic probe with an aligned needle guide system, comparable to the ultrasonic probe already explained above according to DE 699 13 936 T2.

US 2010/0168567 A1 discloses a device and a method for protecting body cavity walls during thermal ablation of tissue in the vicinity of this body cavity. For this purpose, a sheath made of elastic material through which a warming liquid can flow is fitted around the ultrasonic probe and which sheath also has acoustic properties for sound transmission. The additional sheathing comprises a vent opening in the distal ultrasonic probe region, through which air pockets between the sheathing and the ultrasonic probe can escape.

EP 1 455 885 B1 discloses a catheter balloon for the therapeutic modification and positioning of tissue, which is arranged proximal to the distal tip of the catheter and, when dilated, projects radially beyond the catheter in order to position it within the vessel by supporting it on the vessel wall.

All known ultrasound rod probes suitable for performing rectal or vaginal examinations, in particular for the purpose of prostate detection and measurement, have the disadvantage that there is a high risk of mechanical injury to the vessel wall, particularly during the insertion procedure, which to date can only be reduced by the great experience and sensitivity of the treating surgeon.

When using a focused. high-pressure water jet to ablate the prostate tissue, the pelvic floor is also mechanically fixed by the endoscope inserted via the urethra, so that the mobility and thus also the possibility of evasion of the anatomical structures, including the rectum of the male pelvis, is significantly restricted. This increases the risk of injury when inserting a transrectal ultrasound rod probe.

### DESCRIPTION OF THE INVENTION

The object of the invention is to further develop an arrangement with an ultrasound rod probe for carrying out rectal or vaginal examinations with a distally rounded probe head and in which at least one ultrasound transducer is integrated, so that the risk of injury explained above is to be significantly reduced, in particular during intracorporeal insertion and positioning of the ultrasound probe.

The object of the invention is solved by an ultrasonic probe according to claim 1. Advantageously further developing features of the invention can be found in the dependent claims and with reference to the following description, in particular to the exemplary embodiments illustrated in the figures.

The arrangement according to the solution having an ultrasonic rod probe for carrying out rectal or vaginal examinations with a distally rounded probe head with the features of the preamble of claim 1 is characterized in that at least one pressure-sensitive element is attached to the probe head, which element undergoes deformation in response to mechanical contact with an object when a pressure acting between the pressure-sensitive element and the object is exceeded.

The deformation properties of the pressure-sensitive element are to be predetermined in such a way that the pressure-sensitive element can be deformed by the resulting resistance force when interacting with the object, which in the specific application case corresponds to a biological vessel wall, in particular the intestinal wall, before any mechanically induced damage occurs to the object, i.e. the intestinal wall. The pressure-sensitive element, which preferably protrudes distally above the sensor head, can preferably be assigned a modulus of elasticity. or e-modulus for short, which is lower or smaller than a modulus of elasticity that can be assigned to the object, preferably the intestinal wall, so that the risk of injury can be considerably reduced simply by selecting a suitable material for the pressure-sensitive element.

In addition, in order to provide the surgeon with timely knowledge of a possible imminent risk of damage or injury to the bowel wall. the event of the incipient and complete deformation of the pressure-sensitive element must be conveyed to the surgeon in a physiologically perceptible manner, so that the surgeon varies or interrupts the insertion process of the ultrasound rod probe. This physiological recognition by the surgeon can take place in different ways. In the simplest case, the person perceives the contact and the associated elastic deformation of the pressure-sensitive element due to the resistance force by a mere increase in the thrust force acting along the ultrasonic rod probe and required for the insertion process. In this case, the person can interrupt the insertion process. However, this presupposes that the pressure-sensitive element has a length that exceeds the probe head distally in order to ensure a sufficiently long deformation path for the pressure-sensitive element for the person and their average reaction time. A preferred embodiment in this regard provides an elastomer means attached to the distal end of the sensor head, preferably in the form of a web-like, elastically bendable extension that projects distally beyond the probe head. The elastomer means typically has a maximum length of 3 cm.

As an alternative to or in combination with the above-mentioned elastomer means, a gel pad or gel body attached to the distal probe head serves as a pressure-sensitive element through which the contact event between the distal probe head and an intracorporeal object, preferably the intestinal wall, can be physiologically perceived by the person through a noticeable increase in thrust force even before intracorporeal damage occurs. In addition to the increasing thrust force upon contact between the probe head and the intestinal wall. both the elastomer means and a gel pad or gel body can be used for gentle and improved sliding of the probe head along the intestinal wall, in particular along a fold of the bowel.

A further alternative for forming a pressure-sensitive element and attaching it to the probe head is an electrically operable pressure sensor, the sensor signals of which can be transmitted to a signaling means arranged proximally on the ultrasonic rod probe, which is capable of generating an optical, acoustic and/or haptically perceptible signal that attracts the person's attention.

Regardless of the type and design of the at least one pressure-sensitive element, it is advisable to inseparably integrate the at least one pressure-sensitive element as an integral component in or on the probe head or to detachably attach it firmly to the probe head in a suitably selected fastening means. A preferred fastening means is at least one elastomeric condom-like cover which surrounds the probe head and in which the at least one pressure-sensitive element is integrated or between which and the probe head the at least one pressure-sensitive element is arranged.

### BRIEF DESCRIPTION OF THE INVENTION

Without limiting the general idea of the invention, the invention is described below by way of exemplary embodiments with reference to the drawing. In particular:

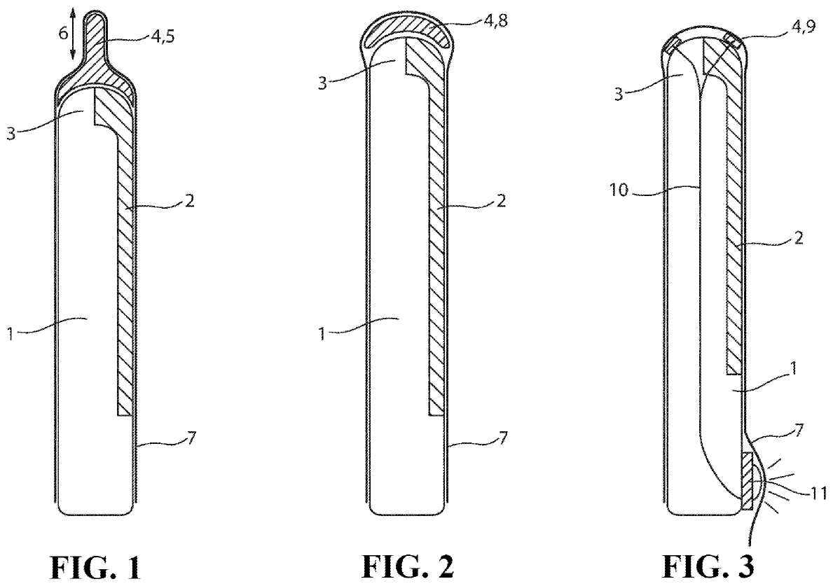

FIG. 1 shows an exemplary embodiment with elastically bendable element on the probe head,

FIG. 2 shows an exemplary embodiment with gel pad on the probe head and

FIG. 3 shows an exemplary embodiment with a pressure sensor on the probe head.

### WAYS OF CARRYING OUT THE INVENTION, INDUSTRIAL APPLICABILITY

FIG. 1 schematically shows the distal region of an ultrasonic rod probe 1 with at least one ultrasonic transducer 2. The ultrasonic rod probe 1 has a rounded probe head 3 to which a pressure-sensitive element 4 in the form of an elastomer nipple 5 is attached. The elastomer nipple 5 has a length 6 that projects distally beyond the probe head 3 and should preferably not exceed three centimeters. The elastomer nipple 5 can have a web-like or rib-like design or a shape that deviates from this but does not counteract the flexibility of the elastic modulus inherent in the elastomer material. In the exemplary embodiment shown, the nipple shape of the pressure-sensitive element 4 widens from its distal end in the direction of the probe head 3 and takes approximately the diameter of the probe head.

To attach the elastomer nipple 5 to the probe head 3, it is possible on the one hand to join the elastomer nipple 5 directly to the probe head 5 by means of an adhesive bond. Alternatively, it is possible to fit the elastomer nipple 5 loosely to the probe head 3 and to join it firmly relative to the probe head 3 with the aid of a condom-like cover 7, the length, shape and diameter of which is essentially adapted to the distal region of the ultrasonic rod probe 1.

It is also conceivable to form the elastomer nipple 5 as an integral unit with the condom-like cover 7, which must be slipped over the ultrasonic rod probe 1 before it is used.

The pressure-sensitive element 4, which is designed as an elastomer nipple 5, ensures that when the ultrasonic rod probe 1 is advanced along the intestine, the elastomer nipple 5 pushes the intestinal wall away when it comes into contact with, for example, a Kohlrausch bend and also pushes the ultrasonic rod probe 1 away from the intestinal wall as it is advanced further through the intestine.

In FIG. 2, instead of an elastomer nipple 5, a gel pad 8 is arranged on the probe head 3 as a pressure-sensitive element 5. The gel pad 8 corresponds to a soft, pliable mass, typically in the form of soft silicone or a gel-filled pouch. When the soft mass comes into contact, it deforms when pressed against an intracorporeal resistance, e.g. a Kohlrausch bend, which pushes the ultrasonic rod probe 1 away from it.

The gel pad 8 can be attached directly or indirectly to the probe head 3 in the same way as the elastomer nipple 5 described above.

FIG. 3 shows an embodiment in which the pressure-sensitive element 4 is in the form of one or more pressure sensors 9. The at least one pressure sensor 9 is preferably arranged as an integral component in or on the probe head 3 of the ultrasonic rod probe 1. For the electrical supply and output of the sensor signals, the pressure sensor 9 is connected via an electrical signal line 10 both to an energy and control unit not shown further, but in particular also to a signal means 11 arranged proximally on the ultrasonic rod probe 1. When the pressure sensor 9 is activated, a signal is transmitted to and activates the signal means 11 arranged proximally, whereby the person operating the ultrasonic rod probe 1 is alerted by perceiving a visual, acoustic and/or haptically perceptible signal.

A purely mechanical pressure pick-up and pressure-dependent proximally directed signal transmission for alerting the person is also conceivable. Such a purely mechanical pressure system comprises a liquid reservoir arranged on the probe head 3, which is pressure-sensitive and when a distally oriented pressure occurs, a preferably colored liquid is pressed through a tube running proximally along the ultrasonic rod probe 1 and is detected extracorporeally by the person.

### LIST OF REFERENCE NUMERALS

1 Ultrasonic rod probe
2 Ultrasonic transducer
3 Probe head
4 Pressure-sensitive element
5 Elastomer nipple
6 Length of the elastomer nipple
7 Cover
8 Gel pad
9 Pressure sensor
10 Signal line
11 Signal means

1. An arrangement with an ultrasound rod probe for carrying out rectal or vaginal examinations with a probe head rounded on the distal side, at least in which an ultrasound transducer is integrated, wherein at least one pressure-sensitive element is attached to the probe head, which is subject to deformation by mechanical contact with an object when a pressure acting between the pressure-sensitive element and the object is exceeded.

2. The arrangement according to claim 1, wherein the at least one pressure-sensitive element is one of the following elements: pressure sensor, gel cushion, elastomer nipple.

3. The arrangement according to claim 1, wherein the at least one pressure-sensitive element is integrated in or on the probe head.

4. The arrangement according to claim 1, wherein an elastomeric condom-like covering is provided which encloses at least the probe head.

5. The arrangement according to claim 4, wherein the at least one pressure-sensitive element is integrated into the cover.

6. The arrangement according to claim 4, wherein the at least one pressure-sensitive element is arranged between the probe head and the coating.

7. The arrangement according to claim 1, wherein the at least one pressure sensitive element has an E-modulus which is smaller than an E-modulus which corresponds to a biological vessel wall.

8. The arrangement according to claim 2, wherein the elastomer nipple is designed in the manner of a web-like, flexible extension which projects distally beyond the probe head.

9. The arrangement according to claim 8, wherein the elastomer nipple has a rounded distal end and a length of up to 3 cm projecting distally beyond the probe head.

10. The arrangement according to claim 2, wherein the pressure sensor is connected to a signaling means arranged on the proximal side of the ultrasound rod probe, which is capable of generating an optical, acoustic and/or haptically perceptible signal.
